(19) United States
(12) Patent Application Publication
(10) Pub. No.: US 2022/0218973 A1
(43) Pub. Date: Jul. 14, 2022

(54) Connector for In-Line Selective Occlusion of Drainage Tube

(71) Applicant: C. R. Bard, Inc., Franklin Lakes, NJ (US)

(72) Inventors: Varad Chavan, Kolhapur (IN); Rohit Sinha, Lawrenceville, GA (US)

(21) Appl. No.: 17/571,254

(22) Filed: Jan. 7, 2022

Related U.S. Application Data
(60) Provisional application No. 63/135,480, filed on Jan. 8, 2021.

(51) Int. Cl. A61M 39/10; A61M 39/24

(52) U.S. Cl. CPC A61M 39/10; A61M 25/0017; A61M 39/24

(57) ABSTRACT

Embodiments disclosed herein are directed to a connector for a fluid drainage system for fluidly coupling a catheter to a drainage tube. The connector includes a body defining a drainage lumen extending along a longitudinal axis from a distal portion to a proximal portion. The connector also includes a positive air pressure inlet and a valve slidably engaged with a valve housing along the longitudinal axis between a first position and a second position. The valve in the first position provides fluid communication between the distal portion and the proximal portion of the drainage lumen, and the valve in the second position occludes fluid communication between the proximal portion and the distal portion of the drainage lumen. Pressurized air provided at the inlet can transition the valve to the second position and clear any dependent loops within the drainage lumen.

### PRIORITY

This application claims the benefit of priority to U.S. Provisional Application No. 63/135,480, filed Jan. 8, 2021, which is incorporated by reference in its entirety into this application.

### SUMMARY

Briefly summarized, embodiments disclosed herein are directed to a valved connector for in-line selective occlusion of a drainage lumen to prevent pressure reflux during active clearance of dependent loops within fluid drainage systems.

Fluid drainage systems generally include a flexible drainage tube configured to provide fluid communication with a collection container. Due to the flexibility of the drainage tube, and/or normal patient movements, sections of positive incline can form, where drainage fluid can accumulate, termed “dependent loops.” Fluid caught in these dependent loops can lead to various problems. For example, fluid caught in the drainage tube fails to reach the collection container leading to inaccurate fluid output measurements and misdiagnosis of patients or mis-prescribing of drugs. For dependent loops in urine drainage systems, the bladder must push against the pressure of the dependent loop to further excrete urine. This can be uncomfortable for the patient and can lead to injury if the pressure is not alleviated in a timely manner. Further, stagnant fluid within the drainage tube can be a source of pathogens leading to an increased risk of catheter associated urinary tract infections (CAUTI). CAUTI can be highly detrimental to the patient as well as incurring increased costs for additional treatment.

Current practice is for clinicians to manipulate the tubing to urge the fluid caught in the dependent loop towards the collection container. If performed incorrectly, fluid reflux can occur causing infections and complications. Further, there is also an added responsibility on the clinician to perform the manipulation correctly and in a timely manner. Active drainage systems have been developed that introduce a positive air flow to a distal end of the drainage tube to urge fluid through the system to the collection container, clearing these dependent loops. However, the air pressure within the system can cause increased pressure within the patient bladder leading to reflux, increased discomfort and potentially introducing infections to the patient.

Disclosed herein is a connector for a fluid drainage system for coupling a catheter to a drainage tube including, a body defining a drainage lumen extending along a longitudinal axis from a distal portion to a proximal portion, a positive air pressure inlet, and a valve slidably engaged with a valve housing along the longitudinal axis between a first position and a second position wherein, the valve in the first position provides fluid communication between the distal portion and the proximal portion of the drainage lumen, and the valve in the second position occludes fluid communication between the proximal portion and the distal portion of the drainage lumen.

In some embodiments, the valve includes a valve plate defining a proximal face and a distal face, each extending perpendicular to the longitudinal axis, the distal face configured to engage a proximal end of the body in the second position and create a fluid tight seal therebetween. The valve includes one or more legs extending distally from the distal face and configured to slidably engage an inner surface of a valve recess disposed in the proximal end of the body. The leg further includes a pawl disposed at a distal end thereof and configured to engage a lip extending radially inwards from a rim of the valve recess, the pawl configured to prevent further proximal movement of the valve when in the first position.

In some embodiments, the connector further includes a biasing member configured to bias the valve towards the first position. In some embodiments, the connector further includes an inlet housing engaged with a proximal end of the valve housing, the inlet housing including the positive air pressure inlet. In some embodiments, an axis of the positive air pressure inlet is angled at 45o relative to the longitudinal axis. In some embodiments, an axis of the positive air pressure inlet is angled at 90o relative to the longitudinal axis. In some embodiments, an axis of the positive air pressure inlet defines an S-shape. The valve housing is formed of a transparent material. In some embodiments, the connector further includes a distal coupling disposed at a distal end of the body and configured to releasably engage a proximal end of the catheter to provide fluid communication between the catheter and the distal portion of the drainage lumen. In some embodiments, the distal coupling is one of a luer slip fit, threaded, spin-nut, interference fit, press-fit, or snap-fit coupling. The catheter is a Foley catheter configured to drain urine from a bladder of a patient.

In some embodiments, the connector further includes a proximal coupling disposed at a proximal end of the connector and configured to engage a distal end of the drainage tube, the drainage tube in fluid communication with a collection container. The proximal coupling is one of a luer slip fit, threaded, spin-nut, interference fit, press-fit, or snap-fit coupling. In some embodiments, the connector further includes a sample port or a pressure sensor port in fluid communication with the distal portion of the drainage lumen. The valve housing is engaged with the body in one of an interference fit, press-fit, snap-fit, or threadable engagement. The valve housing further includes an abutment extending radially inward from an inner surface and configured to abut against the valve in the first position to prevent any further proximal movement of the valve.

Also disclosed is a method of draining a fluid from a catheter to a collection container including, draining a fluid along a longitudinal axis of a connector, from a distal drainage lumen to a proximal drainage lumen, applying a pressurized fluid to an inlet of the connector, sliding a valve along the longitudinal axis from a first position to a second position, occluding fluid flow between the distal drainage lumen and the proximal drainage lumen, and urging the fluid from the proximal drainage lumen to the collection container.

In some embodiments, the method further includes creating a fluid-tight seal between a distal face of a valve plate of the valve, with a proximal end of the body, when the valve is in the second position to occlude fluid flow between the distal drainage lumen and the proximal drainage lumen. In some embodiments, the method further includes engaging a leg of the valve with an inner surface of a valve recess disposed in the proximal end of the body, the leg extending distally from the distal face. In some embodiments, the method further includes engaging a pawl disposed at a distal end of the leg, with a lip extending radially inwards from a rim of the valve recess to prevent further proximal movement of the valve when in the first position. In some embodiments, the method further includes biasing the valve towards the first position. In some embodiments, the method further includes an inlet housing engaged with a proximal end of the valve housing in one of an interference fit, press-fit, snap-fit, or threadable engagement, the inlet housing including the inlet providing fluid communication with the proximal drainage lumen.

In some embodiments, an axis of the inlet is angled at 45o relative to the longitudinal axis. In some embodiments, an axis of the inlet is angled at 90o relative to the longitudinal axis. In some embodiments, an axis of the inlet defines an S-shape. In some embodiments, the valve housing is formed of a transparent material. In some embodiments, the method further includes coupling a distal coupling disposed at a distal end of the body with a proximal end of the catheter to drain a fluid from the catheter to the distal drainage lumen. The distal coupling is one of a luer slip fit, threaded, spin-nut, interference fit, press-fit, or snap-fit coupling. The catheter is a Foley catheter configured to drain urine from a bladder of a patient. In some embodiments, the method further includes coupling a proximal coupling disposed at a proximal end of the connector with a distal end of the drainage tube, the drainage tube in fluid communication with a collection container. In some embodiments, the proximal coupling is one of a luer slip fit, threaded, spin-nut, interference fit, press-fit, or snap-fit coupling.

In some embodiments, the method further includes a sample port or a pressure sensor port in fluid communication with the distal portion of the drainage lumen. The valve housing is engaged with the body in one of an interference fit, press-fit, snap-fit, or threadable engagement. In some embodiments, the method further includes abutting the valve against an abutment in the first position to prevent any further proximal movement of the valve, the abutment extending radially inward from an inner surface of the valve housing.

Also disclosed is a fluid drainage system including, a catheter extending along a longitudinal axis, and a connector fluidly coupled to the catheter having, a body defining a distal drainage lumen, a valve housing defining a proximal drainage lumen, an inlet in fluid communication with the proximal drainage lumen, and a ball valve slidably engaged within the valve housing between a first position and a second position, the ball valve configured to occlude fluid communication between the valve housing and the body when a positive air pressure is provided at the inlet.

In some embodiments, the ball valve is configured to engage a proximal end of the body in the second position and create a fluid tight seal therebetween. In some embodiments, the fluid drainage system further includes an O-ring disposed between the ball valve and the body and configured to create a fluid tight seal therebetween when the ball valve is in the second position. The valve housing includes one or more fins extending radially inward to define a proximal drainage lumen diameter the same as a diameter of the ball valve. In some embodiments, the fluid drainage system further includes an inlet housing engaged with a proximal end of the valve housing, the inlet housing including the inlet extending therefrom. In some embodiments, an axis of the inlet is angled at 45° relative to the longitudinal axis. In some embodiments, an axis of the positive air pressure inlet is angled at 90° relative to the longitudinal axis. In some embodiments, an axis of the positive air pressure inlet defines an S-shape. In some embodiments, the valve housing is formed of a transparent material.

In some embodiments, the fluid drainage system further includes a distal coupling disposed at a distal end of the body and configured to releasably engage a proximal end of the catheter the distal coupling including one of a luer slip fit, threaded, spin-nut, interference fit, press-fit, or snap-fit coupling. In some embodiments, the fluid drainage system further includes a proximal coupling disposed at a proximal end of the connector and configured to engage a distal end of the drainage tube, the drainage tube in fluid communication with a collection container, the proximal coupling including one of a luer slip fit, threaded, spin-nut, interference fit, press-fit, or snap-fit coupling. In some embodiments, the catheter is a Foley catheter configured to drain urine from a bladder of a patient.

Also disclosed is a method of draining a fluid from a catheter to a collection container including, draining a fluid from a body of a connector to a valve housing of the connector, the body defining a distal drainage lumen and the valve housing defining a proximal drainage lumen, applying a pressurized fluid to an inlet of the connector, transitioning a ball valve from a first position disposed in a spaced apart relationship from a proximal end of the body, to a second position where the ball valve is engaged with the proximal end of the body, occluding fluid flow between the valve housing and the body, and urging the fluid from the proximal drainage lumen to the collection container.

In some embodiments, the method further includes creating a fluid-tight seal between the ball valve and the proximal end of the body. In some embodiments, the method further includes an O-ring disposed between the ball valve and the body and configured to create a fluid tight seal therebetween when the ball valve is in the second position. In some embodiments, the valve housing includes one or more fins extending radially inward to define a proximal drainage lumen diameter, the same as a diameter of the ball valve. In some embodiments, the method further includes an inlet housing engaged with a proximal end of the valve housing, the inlet housing including the inlet extending therefrom. In some embodiments, an axis of the inlet is angled between 45o and 90o relative to the longitudinal axis. In some embodiments, the valve housing is formed of a transparent material. In some embodiments, the catheter is a Foley catheter configured to drain urine from a bladder of a patient.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exemplary fluid drainage system, in accordance with embodiments disclosed herein.

FIG. 2 shows a perspective view of a snap fit valve connector in a first position, in accordance with embodiments disclosed herein.

FIG. 3 shows a perspective view of a snap fit valve connector in a second position, in accordance with embodiments disclosed herein.

FIG. 4A shows an exploded view of a snap fit valve connector, in accordance with embodiments disclosed herein.

FIG. 4B shows close up detail of a snap fit valve of FIG. 4A, in accordance with embodiments disclosed herein.

FIG. 5 shows a cross-section view of a snap fit valve connector in a first position, in accordance with embodiments disclosed herein.

FIG. 6 shows a cross-section view of a snap fit valve connector in a second position, in accordance with embodiments disclosed herein.

FIG. 7 shows a perspective view of a spring valve connector in a first position shown in wire frame, in accordance with embodiments disclosed herein.

FIG. 8 shows a side view of a spring valve connector, in accordance with embodiments disclosed herein.

FIG. 9 shows an exploded view of a spring valve connector, in accordance with embodiments disclosed herein.

FIG. 10 shows a cross-sectional view of a spring valve connector, in accordance with embodiments disclosed herein.

FIG. 11 shows a cross-sectional view of a spring valve connector in a first position, in accordance with embodiments disclosed herein.

FIG. 12 shows a cross-sectional view of a spring valve connector in a second position, in accordance with embodiments disclosed herein.

FIG. 13 shows a perspective view of a ball valve connector in a first position with an S-shaped inlet, in accordance with embodiments disclosed herein.

FIG. 14 shows a perspective view of a ball valve connector in a first position with a straight inlet, in accordance with embodiments disclosed herein.

FIG. 15 shows an exploded view of a ball valve connector, in accordance with embodiments disclosed herein.

FIG. 16 shows a lateral cross-section view of the connector shown in FIG. 15, in accordance with embodiments disclosed herein.

FIG. 17 shows a lateral cross-section view of the connector shown in FIG. 15 with the ball bearing, in accordance with embodiments disclosed herein.

FIG. 18 shows a longitudinal cross-section view of a ball valve connector in a first position, in accordance with embodiments disclosed herein.

FIG. 19 shows a longitudinal cross-section view of a ball valve connector in a second position, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

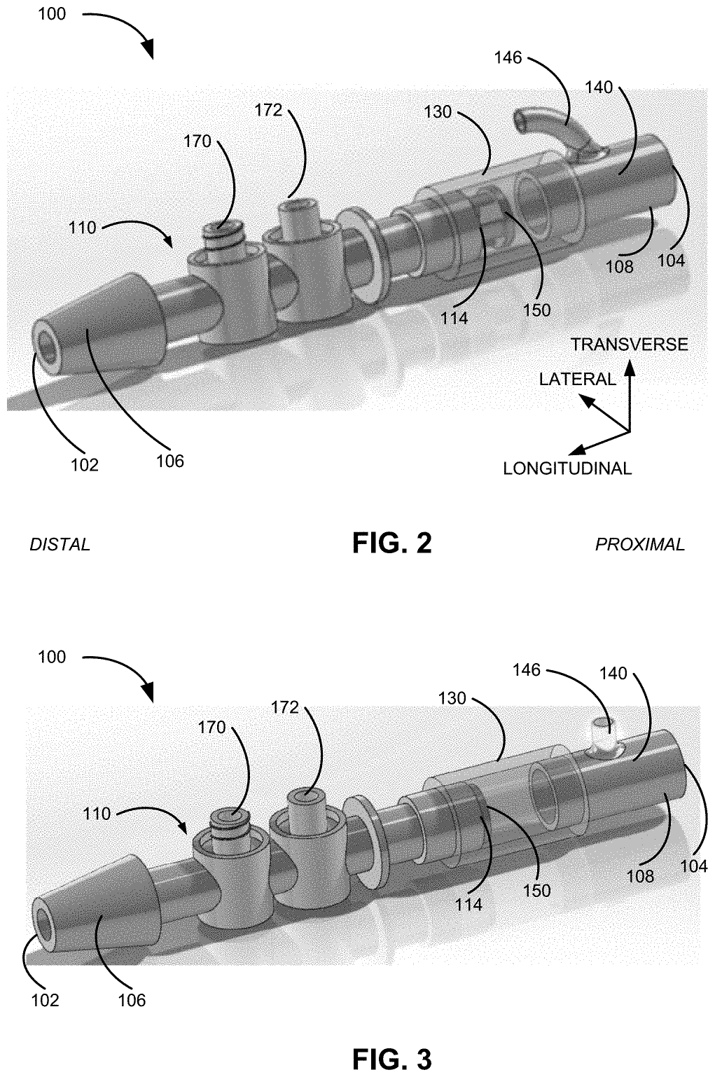

To assist in the description of embodiments described herein, as shown in FIG. 2, a longitudinal axis extends substantially parallel to an axial length of the drainage lumen. A lateral axis extends normal to the longitudinal axis, and a transverse axis extends normal to both the longitudinal and lateral axes.

As used herein, the term “fluid” can include a gas, liquid, or combination thereof. Unless defined otherwise, all technical and scientific terms used herein have the same meaning as commonly understood by those of ordinary skill in the art.

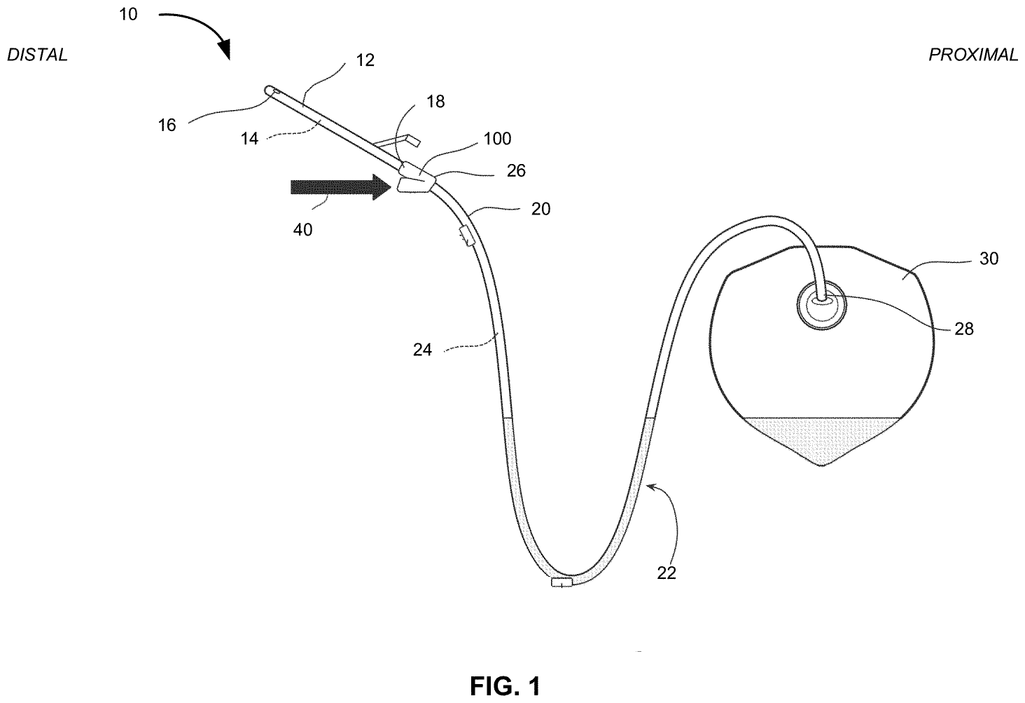

FIG. 1 shows an exemplary drainage system (“system”) 10, configured to drain a fluid from a patient. The system 10 generally includes a catheter 12, a drainage tube (“tube”) 20, and a collection container (“container”) 30. Exemplary catheters 12 include indwelling catheters, Foley catheters, balloon catheters, peritoneal drainage catheters, or the like, and are configured to be inserted into an orifice within the body of a patient to drain a fluid therefrom. Exemplary fluids can include water, blood, plasma, urine, interstitial fluid, saliva, mucus, pus, or the like. In an embodiment, the catheter 12 can be inserted through the urethra and into a bladder of a patient to drain a fluid, e.g. urine, therefrom. However, it will be appreciated that embodiments disclosed herein can be used with various fluid drainage systems. The catheter 12 includes an eyelet 16 that provides fluid communication with a lumen 14 of the catheter 12, and is configured to drain a fluid, e.g. urine, from a patient.

The drainage tube 20 extends from a distal end 26 to a proximal end 28 to define an axial length, and defines a lumen 24. The distal end 26 of the tube 20 can be in fluid communication with a proximal 18 end of the catheter 12. The proximal end 28 of the tube 20 can be in fluid communication with a collection container 30, to provide fluid communication between the lumen 14 of the catheter 12 and the collection container 30. The tube 20 can be formed of rubber, plastic, polymer, silicone, or similar suitable material. The collection container 30 can include a rigid container, a flexible collection bag, or similar suitable container for receiving a fluid, e.g. urine, drained from the catheter 12.

As shown in FIG. 1, the flexibility of the drainage tube 20 can result in sections of the tube 20 providing a positive incline relative to the direction of fluid flow therethrough. These positive incline portions allow dependent loops 22 to form, which can lead to fluid pooling within the tube 20. The fluid caught within the dependent loop 22 can result in various problems, including acting as a source for CAUTI causing agents and pathogens, inaccurate fluid output measurements, mis-diagnoses of patients, or the like.

In an embodiment, a pump or similar device can introduce a positive air pressure 40 into the tube lumen 24 to urge the residual fluid of the dependent loop 22 through the tube lumen 24 and into the collection container 30. Exemplary pumps can include peristaltic pumps, diaphragm pumps, solenoid pumps, compressors, medical air lines, valve pumps, syringes, bellows, reciprocating pumps, combinations thereof, or the like. Before the positive air pressure 40 is introduced, the lumen 14 of the catheter 12 must be isolated to prevent the positive air pressure 40 flowing distally through the catheter 12 and into the patient, causing discomfort or trauma.

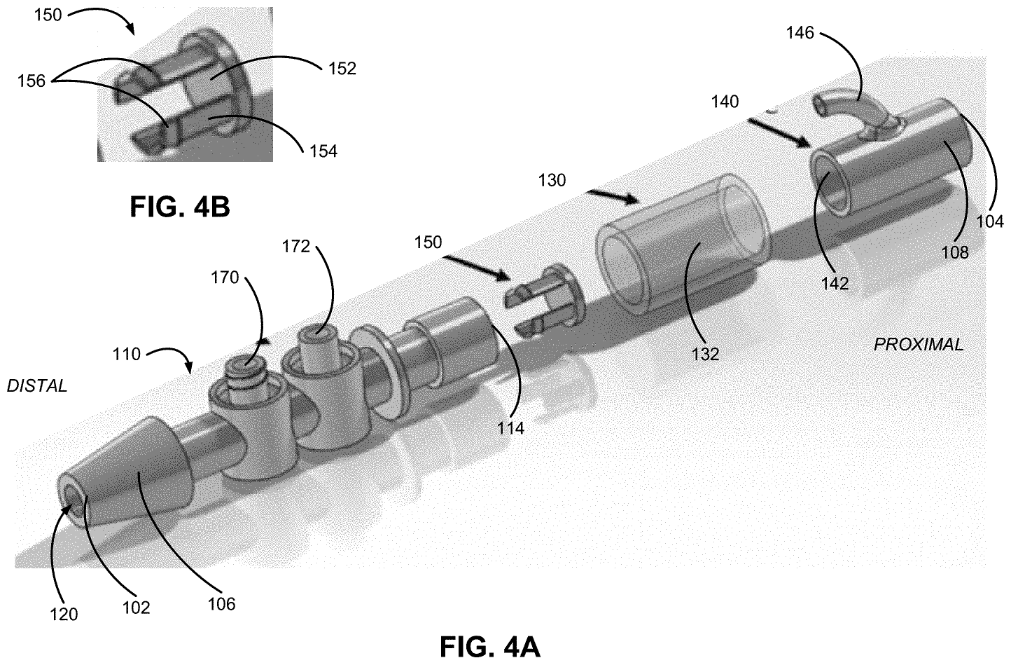

FIGS. 2-6 show various details of an embodiment of a snap-fit valve connector piece (“connector”) 100 disposed between the catheter 12 and the drainage tube 20, and configured to automatically isolate a fluid path communicating with a catheter 12, when a positive air pressure 40 is applied, to clear dependent loops 22. The connector 100 extends along a longitudinal axis from a distal end 102 to a proximal end 104 and generally includes a connector body 110, a valve housing 130, and an inlet housing 140. The connector 100 can further include a drainage lumen 120 extending longitudinally between the distal end 102 and the proximal end 104.

A distal end 102 of the connector 100 can include a distal coupling 106 configured to releasably engage a proximal end of the catheter 12 and provide fluid communication between the lumen 14 of the catheter 12 and the drainage lumen 120 of the connector 100. Similarly, the proximal end 104 of the connector 100 can include a proximal coupling 108 configured to releasably engage a distal end 26 of a drainage tube 20, and provide fluid communication between the drainage lumen 120 of the connector 100 and the lumen 24 of the drainage tube 20. One of the distal coupling 106 or the proximal coupling 108 can include luer slip fit, threaded connector, spin-nut, interference fit, press-fit, snap-fit, or similar connector configured to releasably couple the connector 100 to one of the catheter 12 or the drainage tube 20 with a fluid tight fitting.

The body 110 can define a substantially cylindrical shape extending along a longitudinal axis and can define a distal portion 120A of the drainage lumen 120. A proximal end 114 of the body 110 can include a valve recess 118. In an embodiment, the valve recess 118 can define a larger diameter than the diameter of the drainage lumen, and can be configured to receive a portion of the snap-fit valve (“valve”) 150 therein. The valve 150, or a portion thereof, can be slidably engaged with the valve recess 118 between a first position and a second position, as described in more detail herein.

In an embodiment, the connector 100 can further include a valve housing 130 coupled to a proximal end 114 of the body 110 in an interference fit, press fit, snap fit, or threadable engagement. The valve housing 130 defines a substantially cylindrical shaped valve cavity 132 extending along the longitudinal axis and aligned with the axis of the drainage lumen 120. The valve cavity 132 can define a substantially circular cross-sectional shape, however it will be appreciated that other cross-sectional shapes are also contemplated. The valve cavity 132 can define a portion of the drainage lumen 120. In an embodiment, the valve housing 130 can be formed from a transparent material to allow a user to observe a position of the valve 150 disposed therein, or a flow of fluid therethrough.

In an embodiment, the connector 100 further includes an inlet housing 140 coupled to a proximal end of the valve housing in an interference fit, press fit, snap fit, or threadable engagement. The inlet housing 140 defines a substantially cylindrical shaped inlet cavity 142 extending along the longitudinal axis and aligned with the axis of the drainage lumen 120. The inlet cavity 142 can define a substantially circular cross-sectional shape, however it will be appreciated that other cross-sectional shapes are also contemplated. The inlet cavity 142 can define a portion of the drainage lumen 120. In an embodiment, the valve cavity 132 and the inlet cavity 142 can co-operate to define a proximal portion 120B of the drainage lumen 120B. The inlet housing 140 can include an inlet 146 extending therefrom and configured to provide fluid communication between a pump, or similar source of positive air pressure 40 and the inlet cavity 142. The inlet 146 can include a coupling configured to couple with a positive air pressure fluid line, or the like. Exemplary positive air pressure fluid lines can include, medical air lines, pumps, syringes, or the like.

In an embodiment, the inlet 146 can extend at an angle of between 30° and 90° relative to the longitudinal axis of the drainage lumen 120. In an embodiment, the inlet 146 can extend at substantially 45° relative to the longitudinal axis of the drainage lumen 120. However, greater or lesser angles are also contemplated. In an embodiment, the inlet 146 can define an “S-shaped” axial length. Advantageously, the angle and/or “S-shape” of the inlet 146 can direct the positive air pressure 40 towards the proximal portion 120B of the drainage lumen 120 and into the drainage tube 20, to urge a fluid flow therethrough.

In an embodiment, the connector 100 can further include a sample port 170 communicating with the drainage lumen 120 and extending perpendicular therefrom. In an embodiment, the sample port 170 can include a valve configured to control an access or a fluid flow therethrough. Exemplary valves can include check valves, one way valves, flap valves, duckbilled valves, combinations thereof, or the like. The sample port 170 can be configured to allow a clinician to sample a fluid disposed within the drainage lumen 120. In an embodiment, the body 110 can further include a pressure sensor port 172 communicating with the drainage lumen 120 and extending perpendicular therefrom. In an embodiment, the pressure sensor port 172 can include a pressure sensor configured to detect a fluid pressure within the drainage lumen 120.

In an embodiment, the connector 100 can further include a valve 150, e.g. a “snap-fit valve” 150. The valve 150 can include a plate 152 defining a disc shape and having a diameter that is greater than a diameter of the valve recess 118 at the distal end 114 of the body 110, and less than a diameter of the valve cavity 132. The plate 152 can define a distal face and a proximal face each extending perpendicular to the longitudinal axis. In an embodiment, the valve 150 can include one or more legs 154 extending perpendicular from a distal face of the valve plate 152, i.e. extending distally along a longitudinal axis. As shown the valve 150 includes four legs 154 however, greater or lesser numbers of legs 154 are also contemplated. The leg(s) 154 can slidably engage the valve recess and can maintain the orientation of the plate 152 relative to the longitudinal axis as the valve 150 transitions between the first position and the second position.

In an embodiment, the valve leg 154 can include a pawl 156 disposed at a distal end of the leg 154 and configured to allow the distal end of the leg 154 to advance distally of a lip 158 of the valve recess 118. Further, the pawl 156 can engage the lip 158 in a proximal direction and prevent the distal end of the leg 154 from passing proximally of the lip 158. In an embodiment, the leg 154 can be formed of a resilient material that can allow the leg 154 to flex radially inwards as the pawl 156 is urged distally past the lip 158. The one or more legs 154 can then flex radially outward such that the pawls 156 can engage the lip 158 and prevent any further proximal movement when the valve 150 is in the first position. As such, the valve 150 can engage the valve recess 118 in a snap-fit engagement. Advantageously, the snap-fit valve 150 can provide a simplified construction and assembly for the connector 100 reducing associated manufacturing costs.

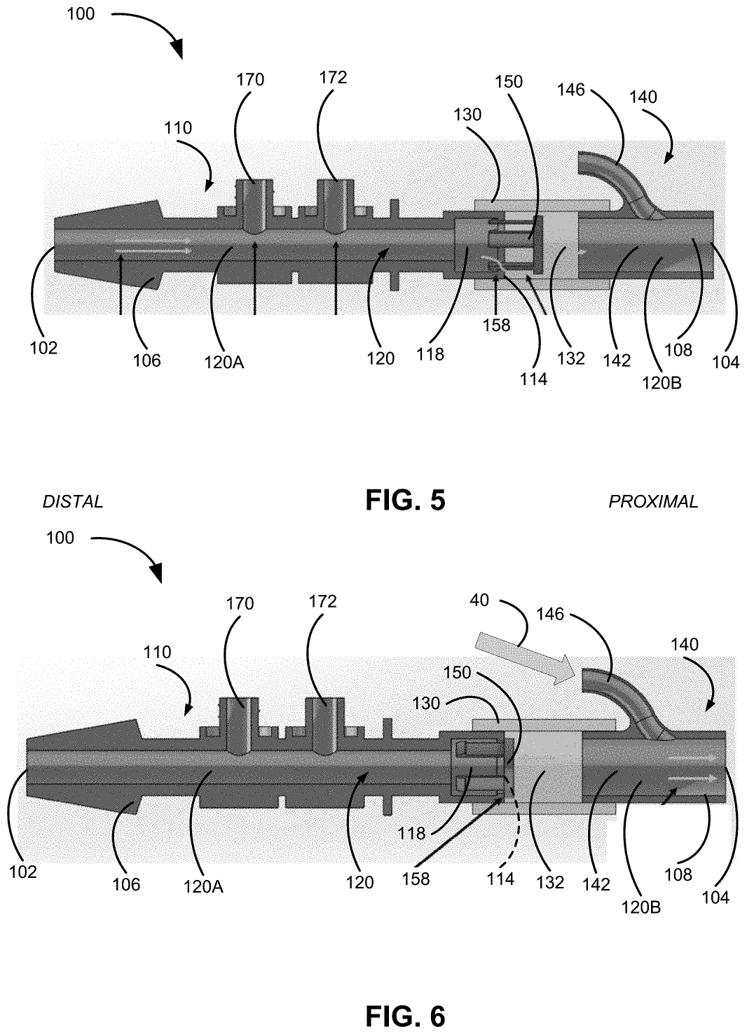

In an embodiment, the valve 150 can be slidable along the longitudinal axis between a first position, as shown in FIG. 5 and a second position, as shown in FIG. 6. In the first position, the valve plate 152 can be positioned in a spaced apart relationship from the proximal end 114 of the connector body 110. The legs 154 can engage the inner surface of the valve recess 118 and maintain the orientation of valve plate 152 relative to the longitudinal axis. Further, the pawls 156 can engage the lip 158 of the valve recess 118 to prevent the valve 150 from sliding further proximally. As such, in the first position, a fluid can flow from the distal portion 120A of the drainage lumen 120, between the legs 154 and past the valve plate into the proximal portion 120B of the connector 100 defined by the valve housing 130 and/or the inlet housing 140.

In an embodiment, a pressurized air 40 can be provided to the inlet 146 and enter the proximal portion 120B of the drainage tube. The increase in air pressure within the proximal portion 120B can transition the valve 150 from the first position (FIG. 5) to the second position (FIG. 6), where the valve plate 152 engages a proximal end 114 of the connector body 110 and creates a fluid tight seal therebetween, preventing any further distal fluid flow. The pressurized fluid 40 can then increase the pressure within proximal portion 120B urging fluid proximally through the drainage lumen 120, through the lumen 24 of the drainage tube 20, and into the collection container 30, clearing any dependent loops 22.

Once the dependent loops 22 have been cleared. The pressurized fluid 40 can be stopped, allowing the pressure within the proximal portion 120B to return to atmospheric pressure. The weight of a fluid flow from the catheter 12 can then flow through the distal portion 120A and transition the valve 150 from the second position (FIG. 6) to the first position (FIG. 5), allowing a fluid to flow proximally into the drainage tube 20.

In an embodiment, a “spring valve” connector piece 200 can include a valve 250 and a biasing member 260 configured to bias the valve 250 towards the first position.

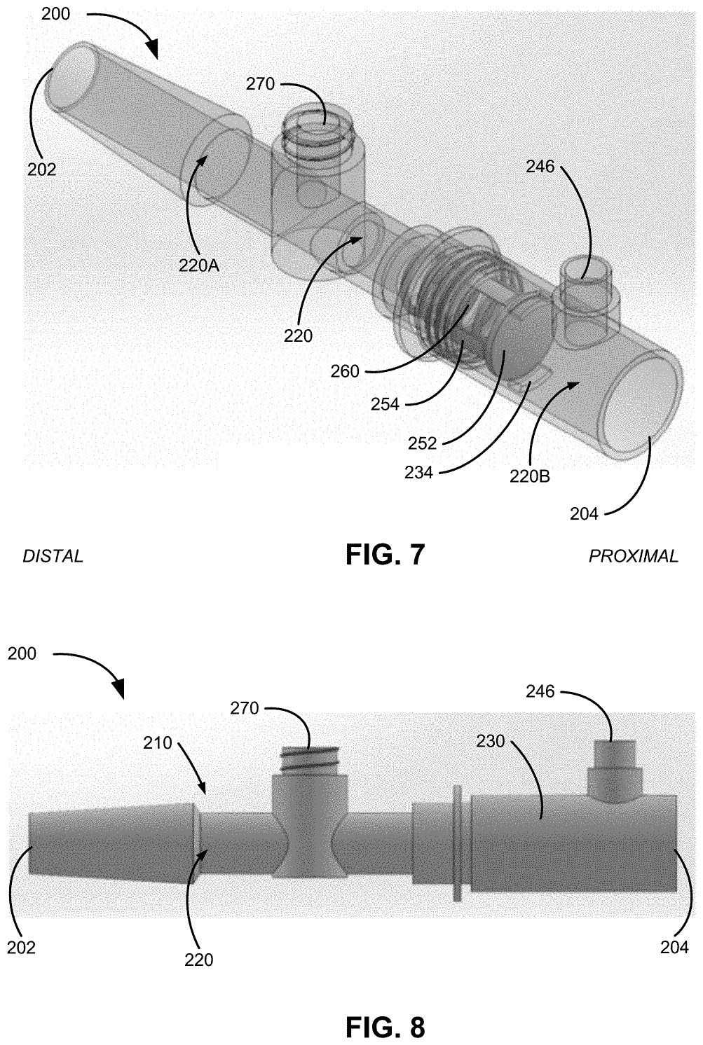

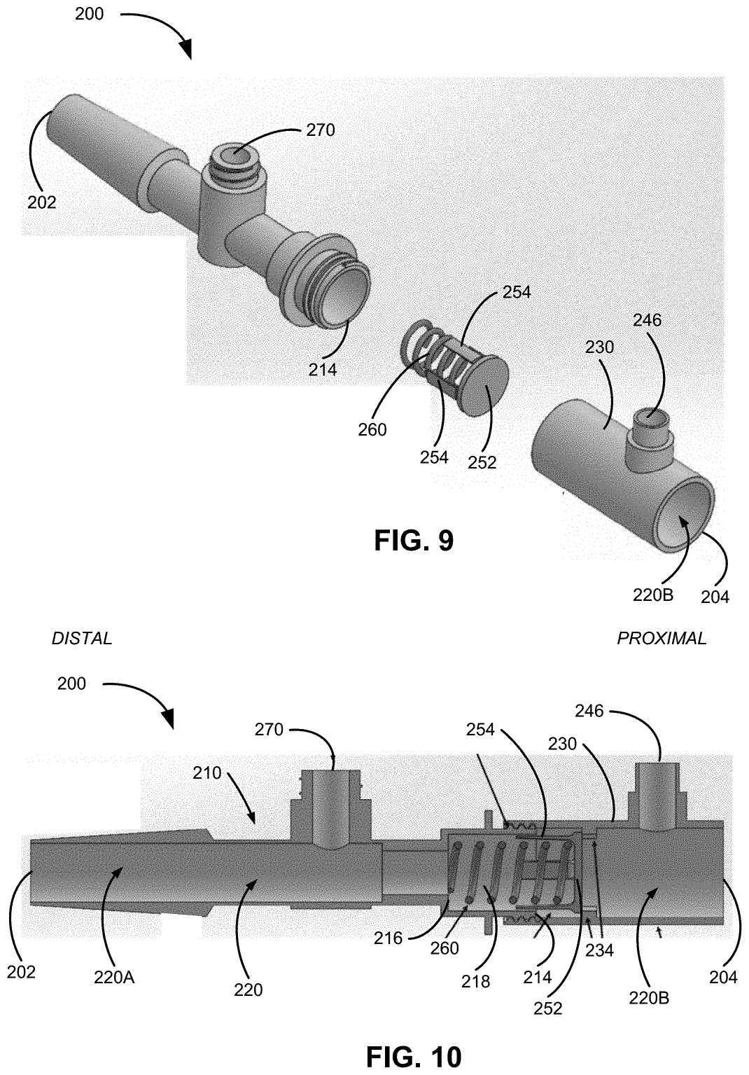

As shown in FIGS. 7-12, the connector 200 can define a drainage lumen 220, extending from a distal end 202 to a proximal end 204, and include a connector body 210 and a valve housing 230, coupled to a proximal end 214 of the body 210. Optionally the connector 200 can include an inlet housing, as described herein. The valve housing 230 can engage the connector body 210 in a threaded engagement, however other forms of attachment are also contemplated including interference fit, press-fit, snap-fit engagements, adhesive, bonding, welding, or the like. In an embodiment, the connector body 210 can define a distal portion 220A of the drainage lumen and the valve housing 230 can define a valve cavity 232 that can define the proximal portion 220B of the drainage lumen 120.

In an embodiment, the valve housing 230 can further include an inlet 246 configured to provide a pressurized fluid 40 to the proximal portion of the drainage lumen 220B. In an embodiment, the inlet 246 can extend at an angle of between 30° and 90° from the valve housing 230. In an embodiment, the inlet 246 can define either a linear axis or a non-linear axis, e.g. an “S-shaped” axis.

In an embodiment, the connector body 210 can include a valve recess 218, disposed at a proximal end thereof. The valve recess 218 can be configured to receive a portion of the valve therein. As described herein, in an embodiment, the valve 250 can include a valve plate 252 defining a proximal side and a distal side, and defining a substantially circular cross-sectional shape, when viewed along a longitudinal axis. The valve 250 can include one or more legs 254 extending distally from a distal side of the plate 252. The legs 254 can slidably engage an inner surface of the recess 218. The legs 254 can maintain alignment of the plate 252 relative to the longitudinal axis, i.e. that a face of the plate 252 extends substantially perpendicular to a longitudinal axis.

In an embodiment, a biasing member 260, e.g. a compression spring, rubber grommet, or the like, can be disposed between the distal face of the plate 252 and a shoulder portion 216 of the recess 218. The spring 260 can bias the valve towards the first position. In an embodiment, the valve housing 230 can further include an abutment 234 extending radially inwards from an inner surface of the valve cavity 232 and configured to abut against the proximal surface of the valve plate 252 when the valve is in the first position. The abutments 234 can prevent the valve 250 from further proximal advancement. To note, the position of the abutments 234 and the length of the legs 254 are configured such that the legs 254 maintain engagement with the recess 218 in both the first position and the second position. The connector body 210 can further include a sample port 270 and/or a pressure sensor port, as described herein.

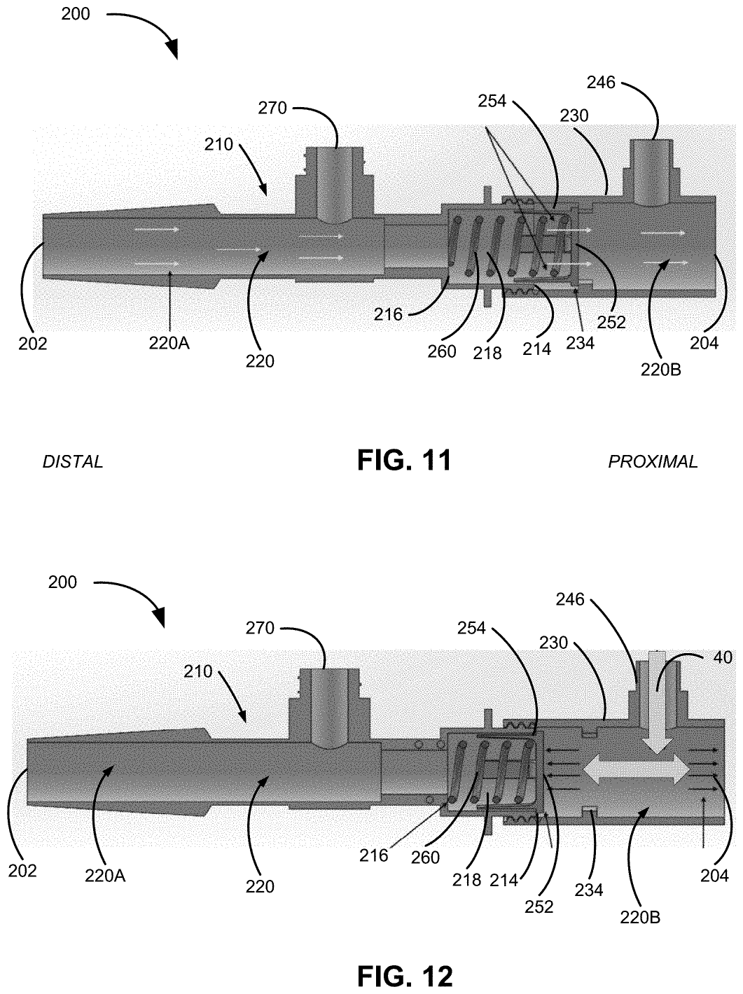

In an exemplary method of use, the connector 200 can provide fluid communication, by way of the drainage lumen 220 between the catheter 12 and the drainage tube 20. As shown in FIG. 11, the biasing member 260 can urge the valve 250 to the first position to allow a fluid to flow from the distal portion 220A, between the legs 254 of the valve 250, and into the proximal portion 220B of the drainage lumen 220 defined by the valve housing 230, and into the drainage tube 20.

As shown in FIG. 12, a positive air pressure can be introduced at the inlet 246. The increase in air pressure within the proximal portion 220B of the drainage lumen 220 can overcome the force of the biasing member 260 and can urge the valve 250 from the first position to the second position. In the second position, the valve plate 252 can engage the proximal end 114 of the connector body 110 to create a fluid tight seal therebetween. As such, the valve plate 252 can prevent any distal fluid flow and allow the pressure within the proximal portion 220B and the drainage tube lumen 24 to build and urge any fluid through the lumen and into the collection container 30, clearing any dependent loops 22.

When the flow of pressurized fluid 40 is ceased, the pressure within the proximal portion 220B reduces, allowing biasing member 260 to transition the valve from the second position to the first position, and allow a fluid flow from the distal portion 220A to the proximal portion 220B. Advantageously, the biasing member 260 can ensure the valve returns to the first position once the air pressure 40 is ceased. This prevents the valve 250 from remaining in the second position after the air pressure 40 is ceased, inhibiting fluid flow from the catheter 12.

In an embodiment, a “ball valve” connector piece 300 can include a ball-bearing valve 350 and, in an embodiment, an O-ring 360. The ball bearing 350 can transition between a first position, to allow a fluid flow between a distal portion 320A and a proximal portion 320B of a drainage lumen 320, and a second position to provide a fluid-tight seal to inhibit a distal fluid flow.

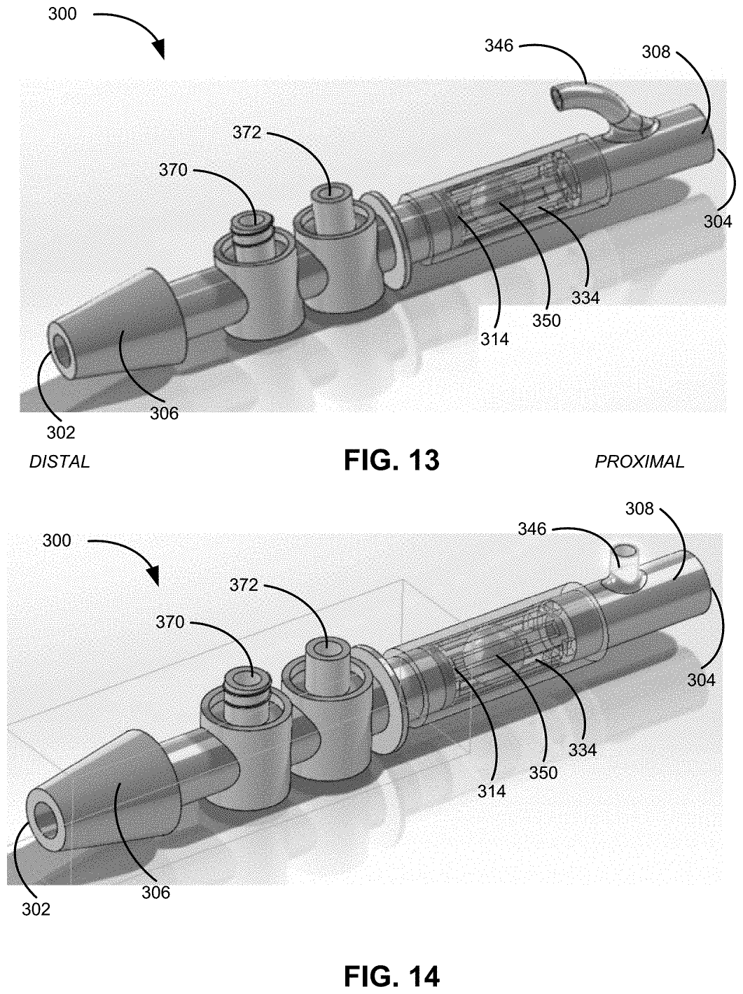

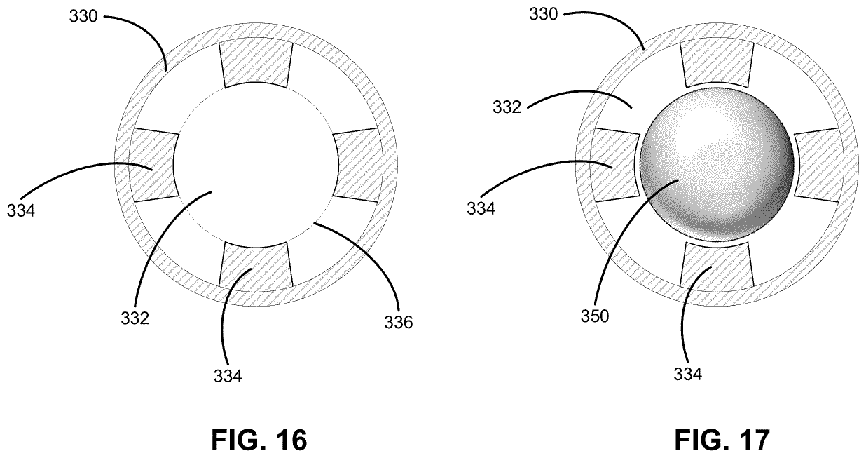

As shown in FIGS. 13-17, the connector 300 can define a drainage lumen 320, extending along a longitudinal axis from a distal end 302 to a proximal end 304. The connector 300 can include a connector body 310, a valve housing 330, and an inlet housing 340. The valve housing 330 can be coupled to a proximal end 314 of the body 310 in an interference fit engagement. Similarly, the inlet housing 330 can be coupled to a proximal end of the valve housing 330 in an interference fit engagement. However other forms of attachment are also contemplated including threaded engagements, press-fit, snap-fit engagements, adhesive, bonding, welding, or the like. The connector body 310 can define a distal portion 320A of the drainage lumen 320. The valve housing 330 can define a valve cavity 332, and the inlet housing 340 can define an inlet cavity 342. The valve cavity 332 and the inlet cavity 342 assembly can define a proximal portion 320B of the drainage lumen 320.

The inlet housing 340 can include an inlet 346 configured to provide a pressurized fluid 40 to the proximal portion of the drainage lumen 320B. In an embodiment, the inlet 346 can extend perpendicular from the valve housing 330. In an embodiment, the inlet 346 can extend at an angle of between 30° and 90° relative to the longitudinal axis of the drainage lumen 320. However, greater or lesser angles are also contemplated. In an embodiment, the inlet 346 can define a linear, non-linear, or “S-shaped” axis. The angle or axial shape of the inlet 346 can be configured to promote a proximal fluid flow through the proximal portion 320B and into the drainage lumen 24.

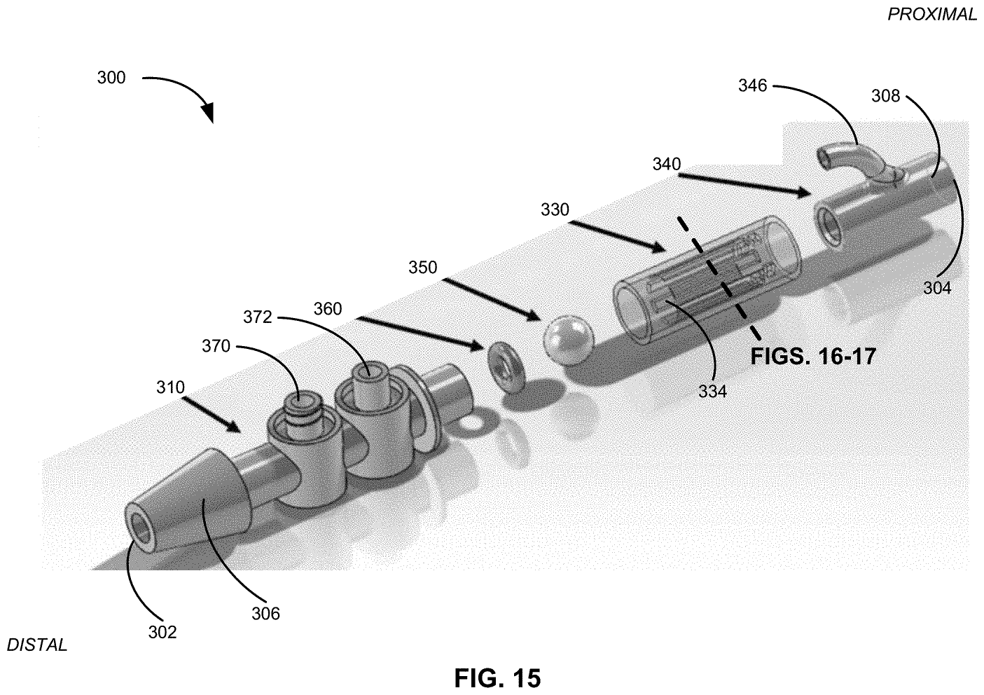

In an embodiment, as shown in FIGS. 15-17, the valve housing cavity 332 can include one or more fins 334 extending radially inward from an inner surface of the valve cavity 332 and extending longitudinally. The fin(s) 334 can extend radially inward to define an inner lumen diameter 336 that is less than the diameter of the valve housing cavity 332. The inner lumen diameter 336 can define a diameter that is the same, or slightly larger than a diameter of the ball bearing 350. As such, the ball bearing 350 can be received within the valve cavity 332 and the fin(s) 334 can maintain the ball bearing 350 in a spaced apart relationship from the inner surface of the valve housing cavity 332. A fluid can flow through the valve housing cavity 332 between the fins 334, the ball bearing 350 and the inner surface of the valve housing cavity 332. In an embodiment, the shape of the fins 334 can reduce a surface contact area between the fin 334 and the ball bearing 350 to reduce friction and facilitate sliding or rolling of the ball bearing 350 between the first position and the second position.

As shown in FIG. 19, in an embodiment, the ball bearing 350 in the second position can engage a proximal end 314 of the connector body 310 and can form a seal therebetween to prevent a distal fluid flow from the proximal portion 320B to the distal portion 320A. The distal end of the connector body 314 can include a beveled or chamfered edge to receive a portion of the ball bearing 350 therein and create a seal.

In an embodiment, the connector 100 can further include an O-ring 360 disposed within valve cavity 332 and engaged with a proximal end 314 of the connector body 310. The O-ring 360 can be formed of a compliant material such as a plastic, polymer, elastomer, rubber, silicone, or the like. The O-ring 360 can be configured to create a fluid-tight seal between the proximal end 314 of the connector body 310 and the ball bearing 350, when the ball bearing 350 is in the second position. In an embodiment, the connector body 310 can further include a sample port 370 and/or a pressure sensor port, as described herein.

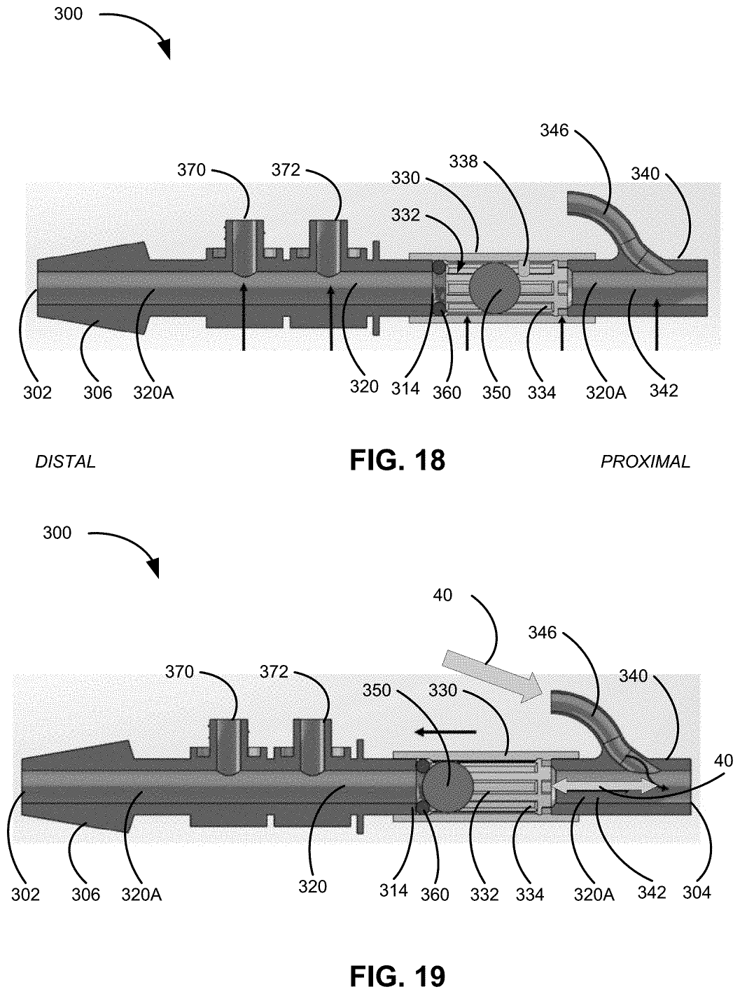

In an exemplary method of use, a connector 300 can be provided, as described herein and can provide fluid communication between the catheter 12 and the drainage tube 20 by way of the drainage lumen 320. As shown in FIG. 18, with the ball bearing 350 in a first position a fluid can flow from the distal portion 320A, through one or more openings between the fin(s) 334 and the ball bearing 350, to the proximal portion 320B.

In an embodiment, the inner lumen diameter 336 defined by the fins 334 can be smaller than a diameter of the ball bearing 350 at a proximal end of the valve housing 330 and can be larger than a diameter of the ball bearing 350 at a proximal end of the valve housing 330. As such the ball bearing 350 can be prevented from engaging the distal end of the inlet housing 340 or creating a seal therebetween. In an embodiment, the valve housing 330 can include an abutment 338 or similar structure, extending radially inward into the inner lumen 336 and configured to prevent the ball bearing 350 from engaging the distal end of the inlet housing 340 and creating a seal therebetween. Advantageously, this can ensure a free proximal flow of fluid through the valve cavity 332, and past the ball bearing 350.

In an embodiment, as shown in FIG. 19, a positive air pressure 40 can be introduced at the inlet 346. The increase in air pressure within the proximal portion 320B of the drainage lumen 320 can urge the ball bearing 350 from the first position (FIG. 18) to the second position (FIG. 19). In the second position, the ball bearing 350 can engage the proximal end 114 of the connector body 110, or the O-ring 360, to create a fluid tight seal therebetween. As such, the ball bearing 350 can prevent any distal fluid flow and allow the pressure within the proximal portion 320B, as well as the drainage tube lumen 24, to build and urge any fluid through the lumen and into the collection container 30, clearing any dependent loops 22.

When the flow of pressurized fluid 40 is ceased, the pressure within the proximal portion 320B reduces, allowing the weight of any fluid within the distal portion 320A to transition the ball bearing 350 from the second position to the first position, and allow a fluid flow to the proximal portion 320B.

Advantageously, embodiments of connectors 100, 200, 300 described herein, transitioning between the first position and the second position automatically isolates the catheter lumen 14 from the pressurized fluid 40 when the pressurized fluid is introduced. The connectors 100, 200, 300 prevent any pressurized fluid 40 from entering the catheter lumen 14, causing trauma or discomfort. Further, the connectors 100, 200, 300 automatically restore patency to the drainage lumen when the pressurized air flow 40 is ceased. This allows fluid to flow from the catheter 12 to the drainage tube 20 preventing fluid build-up distally of the valve 150. Advantageously, embodiments of the connector are configured to prevent accidental isolation of the catheter 12 for any longer than necessary which would quickly lead to fluid buildup and discomfort for the patient. This is important where the patient is incapacitated and cannot notify nursing staff or where the lack of fluid output may go unnoticed.

Further, embodiments of the connector do not require any active control inputs to operate the change in fluid flow paths. Instead the connector only requires the introduction of a positive air pressure 40 to the inlet of the connector. Advantageously, the connector can be used with various positive air pressure dependent loop clearance systems without requiring any communications coupling therebetween, facilitating automation of the dependent loop clearance systems. Moreover, embodiments of the connector do not require any manual input from the clinician to operate, freeing up nursing staff from having to open and close valves in a timely manner.

Advantageously, embodiments of the connector are designed in such a way to provide ease of manufacture and assembly, reducing associated costs. Further, components of the connectors described herein can be easily assembled and disassembled for cleaning and maintenance.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A connector for a fluid drainage system for coupling a catheter to a drainage tube, comprising:
a body defining a drainage lumen extending along a longitudinal axis from a distal portion to a proximal portion;
a positive air pressure inlet; and
a valve slidably engaged with a valve housing along the longitudinal axis between a first position and a second position, wherein:
the valve in the first position provides fluid communication between the distal portion and the proximal portion of the drainage lumen; and
the valve in the second position occludes fluid communication between the proximal portion and the distal portion of the drainage lumen.

2. The connector according to claim 1, wherein the valve includes a valve plate defining a proximal face and a distal face, each extending perpendicular to the longitudinal axis, the distal face configured to engage a proximal end of the body in the second position and create a fluid tight seal therebetween.

3. The connector according to claim 2, wherein the valve includes one or more legs extending distally from the distal face and configured to slidably engage an inner surface of a valve recess disposed in the proximal end of the body.

4. The connector according to claim 3, wherein the leg further includes a pawl disposed at a distal end thereof and configured to engage a lip extending radially inwards from a rim of the valve recess, the pawl configured to prevent further proximal movement of the valve when in the first position.

5. The connector according to claim 1, further including a biasing member configured to bias the valve towards the first position.

6. The connector according to claim 1, further including an inlet housing engaged with a proximal end of the valve housing, the inlet housing including the positive air pressure inlet.

7. The connector according to claim 1, wherein an axis of the positive air pressure inlet is angled at 45° relative to the longitudinal axis.

8. The connector according to claim 1, wherein an axis of the positive air pressure inlet is angled at 90° relative to the longitudinal axis.

9. The connector according to claim 1, wherein an axis of the positive air pressure inlet defines an S-shape.

10. The connector according to claim 1, wherein the valve housing is formed of a transparent material.

11. The connector according to claim 1, further including a distal coupling disposed at a distal end of the body and configured to releasably engage a proximal end of the catheter to provide fluid communication between the catheter and the distal portion of the drainage lumen.

12. The connector according to claim 11, wherein the distal coupling is one of a luer slip fit, threaded, spin-nut, interference fit, press-fit, or snap-fit coupling.

13. The connector according to claim 1, wherein the catheter is a Foley catheter configured to drain urine from a bladder of a patient.

14. The connector according to claim 1, further including a proximal coupling disposed at a proximal end of the connector and configured to engage a distal end of the drainage tube, the drainage tube in fluid communication with a collection container.

15. The connector according to claim 14, wherein the proximal coupling is one of a luer slip fit, threaded, spin-nut, interference fit, press-fit, or snap-fit coupling.

16. The connector according to claim 1, further including a sample port or a pressure sensor port in fluid communication with the distal portion of the drainage lumen.

17. The connector according to claim 1, wherein the valve housing is engaged with the body in one of an interference fit, press-fit, snap-fit, or threadable engagement.

18. The connector according to claim 1, wherein the valve housing further includes an abutment extending radially inward from an inner surface and configured to abut against the valve in the first position to prevent any further proximal movement of the valve.

19. A method of draining a fluid from a catheter to a collection container, comprising:
draining a fluid along a longitudinal axis of a connector, from a distal drainage lumen to a proximal drainage lumen;
applying a pressurized fluid to an inlet of the connector;
sliding a valve along the longitudinal axis from a first position to a second position;
occluding fluid flow between the distal drainage lumen and the proximal drainage lumen; and
urging the fluid from the proximal drainage lumen to the collection container.

20. The method according to claim 19, further including creating a fluid-tight seal between a distal face of a valve plate of the valve, with a proximal end of the body, when the valve is in the second position to occlude fluid flow between the distal drainage lumen and the proximal drainage lumen.

21. The method according to claim 20, further including slidably engaging a leg of the valve with an inner surface of a valve recess disposed in the proximal end of the body, the leg extending distally from the distal face.

22. The method according to claim 21, further including engaging a pawl disposed at a distal end of the leg, with a lip extending radially inwards from a rim of the valve recess to prevent further proximal movement of the valve when in the first position.

23. The method according to claim 19, further including biasing the valve towards the first position.

24. The method according to claim 19, further including an inlet housing engaged with a proximal end of the valve housing in one of an interference fit, press-fit, snap-fit, or threadable engagement, the inlet housing including the inlet providing fluid communication with the proximal drainage lumen.

25. The method according to claim 19, wherein an axis of the inlet is angled at 45° relative to the longitudinal axis.

26. The method according to claim 19, wherein an axis of the inlet is angled at 90° relative to the longitudinal axis.

27. The method according to claim 19, wherein an axis of the inlet defines an S-shape.

28. The method according to claim 19, wherein the valve housing is formed of a transparent material.

29. The method according to claim 19, further including coupling a distal coupling disposed at a distal end of the body with a proximal end of the catheter to drain a fluid from the catheter to the distal drainage lumen.

30. The method according to claim 29, wherein the distal coupling is one of a luer slip fit, threaded, spin-nut, interference fit, press-fit, or snap-fit coupling.

31. The method according to claim 19, wherein the catheter is a Foley catheter configured to drain urine from a bladder of a patient.

32. The method according to claim 19, further including coupling a proximal coupling disposed at a proximal end of the connector with a distal end of the drainage tube, the drainage tube in fluid communication with a collection container.

33. The method according to claim 32, wherein the proximal coupling is one of a luer slip fit, threaded, spin-nut, interference fit, press-fit, or snap-fit coupling.

34. The method according to claim 19, further including a sample port or a pressure sensor port in fluid communication with the distal portion of the drainage lumen.

35. The method according to claim 19, wherein the valve housing is engaged with the body in one of an interference fit, press-fit, snap-fit, or threadable engagement.

36. The method according to claim 19, further includes abutting the valve against an abutment in the first position to prevent any further proximal movement of the valve, the abutment extending radially inward from an inner surface of the valve housing.

37-56. (canceled)
